(19) United States
(12) Patent Application Publication
(10) Pub. No.: US 2024/0003830 A1
(43) Pub. Date: Jan. 4, 2024

(54) IMAGING METHODS USING AN IMAGE SENSOR WITH MULTIPLE RADIATION DETECTORS

(71) Applicant: SHENZHEN XPECTVISION TECHNOLOGY CO., LTD., Shenzhen (CN)

(72) Inventors: Yurun LIU, Shenzhen (CN); Peiyan CAO, Shenzhen (CN)

(21) Appl. No.: 18/368,059

(22) Filed: Sep. 14, 2023

Related U.S. Application Data
(63) Continuation of application No. PCT/CN2021/089135, filed on Apr. 23, 2021.

(51) Int. Cl. G01T 1/29

(52) U.S. Cl. CPC G01T 1/2992; G01T 1/2978; G01T 1/2971

(57) ABSTRACT

Disclosed herein is a method, comprising: capturing portion images of scene portions (i), i=1, . . . , N of a scene with radiation detectors of an image sensor. For i=1, . . . , N, Qi portion images of the scene portion (i) are respectively captured by Qi radiation detectors of the P radiation detectors, Qi being an integer greater than 1. The Qi portion images are of the portion images. The method further includes, for i=1, . . . , N, generating an enhanced portion image (i) from the Qi portion images of the scene portion (i). Generating the enhanced portion image (i) is based on positions and orientations of the Qi radiation detectors with respect to the image sensor and displacements between Qi imaging positions of the scene with respect to the image sensor. The scene is at the Qi imaging positions when the Qi radiation detectors respectively capture the Qi portion images.

### BACKGROUND

A radiation detector is a device that measures a property of a radiation. Examples of the property may include a spatial distribution of the intensity, phase, and polarization of the radiation. The radiation may be one that has interacted with an object. For example, the radiation measured by the radiation detector may be a radiation that has penetrated the object. The radiation may be an electromagnetic radiation such as infrared light, visible light, ultraviolet light, X-ray, or γ-ray. The radiation may be of other types such as α-rays and β-rays. An imaging system may include an image sensor having multiple radiation detectors.

### SUMMARY

Disclosed herein is a method, comprising: capturing M portion images of N scene portions (scene portions (i), i=1, . . . , N) of a scene with P radiation detectors of an image sensor, wherein M, N, and P are positive integers, and wherein for i=1, . . . , N, Qi portion images of the scene portion (i) are respectively captured by Qi radiation detectors of the P radiation detectors, Qi being an integer greater than 1, and wherein the Qi portion images are of the M portion images; and for i=1, . . . , N, generating an enhanced portion image (i) from the Qi portion images of the scene portion (i), wherein said generating the enhanced portion image (i) is based on (A) positions and orientations of the Qi radiation detectors with respect to the image sensor, and (B) displacements between Qi imaging positions of the scene with respect to the image sensor, wherein the scene is at the Qi imaging positions when the Qi radiation detectors respectively capture the Qi portion images.

In an aspect, at least 2 portion images of the M portion images are captured simultaneously by the image sensor.

In an aspect, said at least 2 portion images are captured by at least 2 radiation detectors of the P radiation detectors.

In an aspect, for i=1, . . . , N, Qi>2.

In an aspect, N>1.

In an aspect, Qi=P for i=1, . . . , N.

In an aspect, said generating the enhanced portion image (i) comprises applying one or more super resolution algorithms to the Qi portion images.

In an aspect, said applying the one or more super resolution algorithms to the Qi portion images comprises aligning the Qi portion images.

In an aspect, the method further comprises stitching the enhanced portion images (i), i=1, . . . , N resulting in a stitched image of the scene.

In an aspect, said stitching is based on a position and an orientation of at least one of the P radiation detectors with respect to the image sensor.

In an aspect, the method further comprises determining said displacements between the Qi imaging positions with a step motor which comprises mechanism for measuring a distance of movement caused by the step motor.

In an aspect, the method further comprises determining said displacements between the Qi imaging positions with optical diffraction.

In an aspect, said capturing comprises moving the scene on a straight line with respect to the image sensor throughout said capturing.

In an aspect, the scene does not reverse direction of movement throughout said capturing.

In an aspect, N>1, j and k belong to 1, . . . , N, j≠k, and the Qj radiation detectors are different than the Qk radiation detectors.

In an aspect, N>1, j and k belong to 1, . . . , N, and j≠k, and Qi≠Qk.

Disclosed herein is a method, comprising: capturing M portion images of N scene portions (scene portions (i), i=1, . . . , N) of a scene with P radiation detectors of an image sensor, wherein M, N, and P are positive integers, and wherein for i=1, . . . , N, Qi portion images of the scene portion (i) are respectively captured by Qi radiation detectors of the P radiation detectors, Qi being an integer greater than 1, and wherein the Qi portion images are of the M portion images; and for i=1, . . . , N, generating an enhanced portion image (i) from the Qi portion images of the scene portion (i).

In an aspect, said generating the enhanced portion image (i) is based on (A) displacements and relative orientations between the Qi radiation detectors with respect to the image sensor, and (B) displacements between Qi imaging positions of the scene with respect to the image sensor, wherein the scene is at the Qi imaging positions when the Qi radiation detectors respectively capture the Qi portion images.

In an aspect, at least 2 portion images of the M portion images are captured simultaneously by the image sensor.

In an aspect, said at least 2 portion images are captured by at least 2 radiation detectors of the P radiation detectors.

### BRIEF DESCRIPTION OF FIGURES

FIG. 1 schematically shows a radiation detector, according to an embodiment.

FIG. 2A schematically shows a simplified cross-sectional view of the radiation detector, according to an embodiment.

FIG. 2B schematically shows a detailed cross-sectional view of the radiation detector, according to an embodiment.

FIG. 2C schematically shows an alternative detailed cross-sectional view of the radiation detector, according to an embodiment.

FIG. 3 schematically shows a top view of a package including the radiation detector and a printed circuit board (PCB), according to an embodiment.

FIG. 4 schematically shows a cross-sectional view of an image sensor, where a plurality of the packages of FIG. 3 are mounted to a system PCB, according to an embodiment.

FIG. 5A-FIG. 5N schematically show an imaging process, according to an embodiment.

FIG. 6A-FIG. 6B schematically show an image alignment process, according to an embodiment.

FIG. 7 is a flowchart summarizing and generalizing the imaging process, according to an embodiment.

FIG. 8 is another flowchart summarizing and generalizing the imaging process, according to another embodiment.

### DETAILED DESCRIPTION

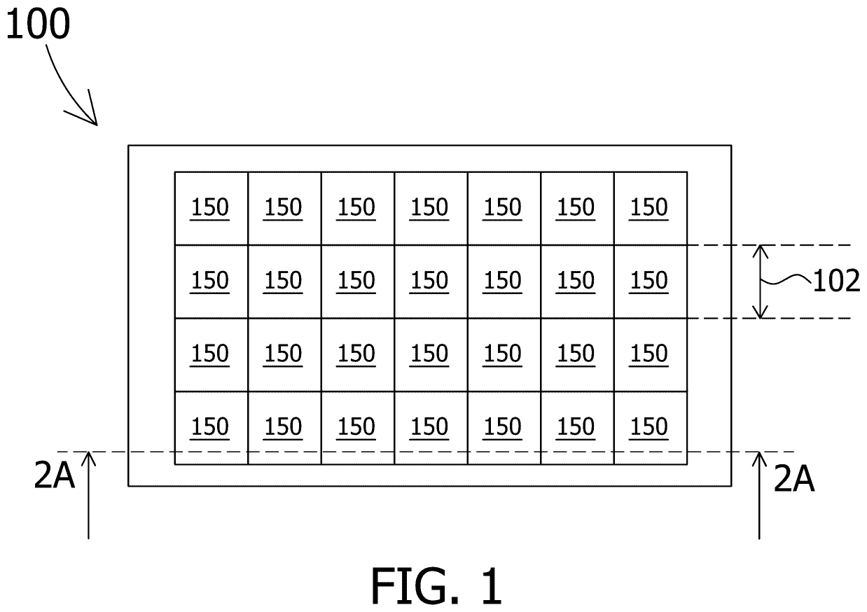

FIG. 1 schematically shows a radiation detector 100, as an example. The radiation detector 100 includes an array of pixels 150 (also referred to as sensing elements 150). The array may be a rectangular array (as shown in FIG. 1), a honeycomb array, a hexagonal array or any other suitable array. The array of pixels 150 in the example of FIG. 1 has 4 rows and 7 columns; however, in general, the array of pixels 150 may have any number of rows and any number of columns.

Each pixel 150 may be configured to detect radiation from a radiation source (not shown) incident thereon and may be configured to measure a characteristic (e.g., the energy of the particles, the wavelength, and the frequency) of the radiation. A radiation may include particles such as photons (electromagnetic waves) and subatomic particles. Each pixel 150 may be configured to count numbers of particles of radiation incident thereon whose energy falls in a plurality of bins of energy, within a period of time. All the pixels 150 may be configured to count the numbers of particles of radiation incident thereon within a plurality of bins of energy within the same period of time. When the incident particles of radiation have similar energy, the pixels 150 may be simply configured to count numbers of particles of radiation incident thereon within a period of time, without measuring the energy of the individual particles of radiation.

Each pixel 150 may have its own analog-to-digital converter (ADC) configured to digitize an analog signal representing the energy of an incident particle of radiation into a digital signal, or to digitize an analog signal representing the total energy of a plurality of incident particles of radiation into a digital signal. The pixels 150 may be configured to operate in parallel. For example, when one pixel 150 measures an incident particle of radiation, another pixel 150 may be waiting for a particle of radiation to arrive. The pixels 150 may not have to be individually addressable.

The radiation detector 100 described here may have applications such as in an X-ray telescope, X-ray mammography, industrial X-ray defect detection, X-ray microscopy or microradiography, X-ray casting inspection, X-ray non-destructive testing, X-ray weld inspection, X-ray digital subtraction angiography, etc. It may be suitable to use this radiation detector 100 in place of a photographic plate, a photographic film, a PSP plate, an X-ray image intensifier, a scintillator, or another semiconductor X-ray detector.

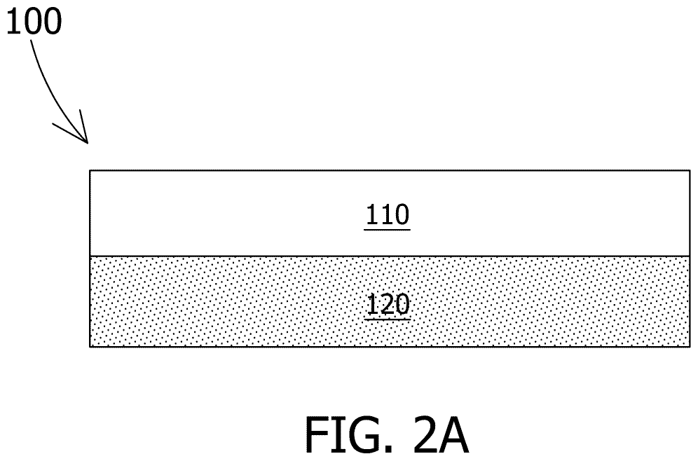

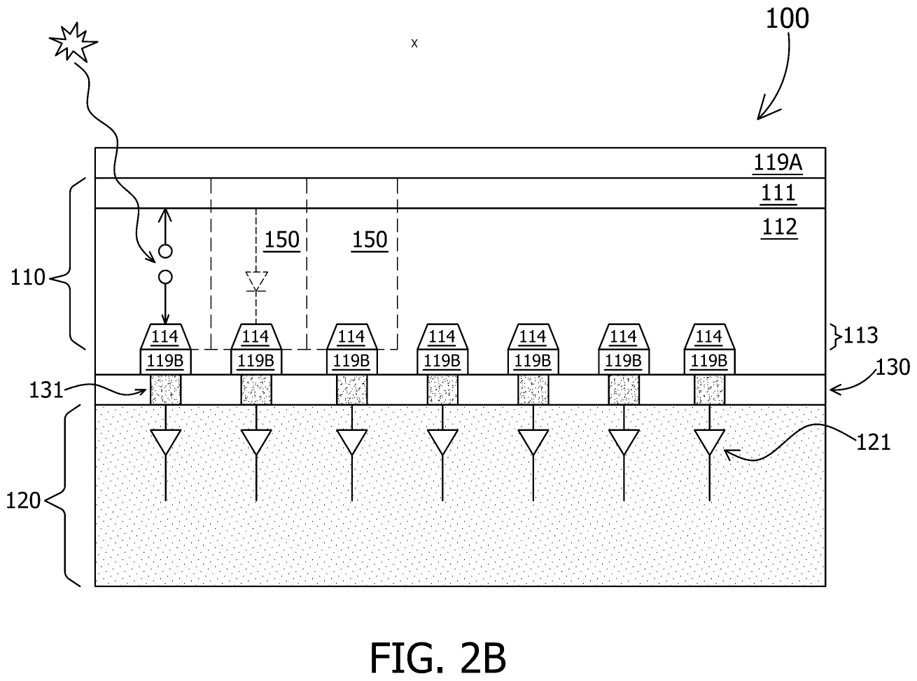

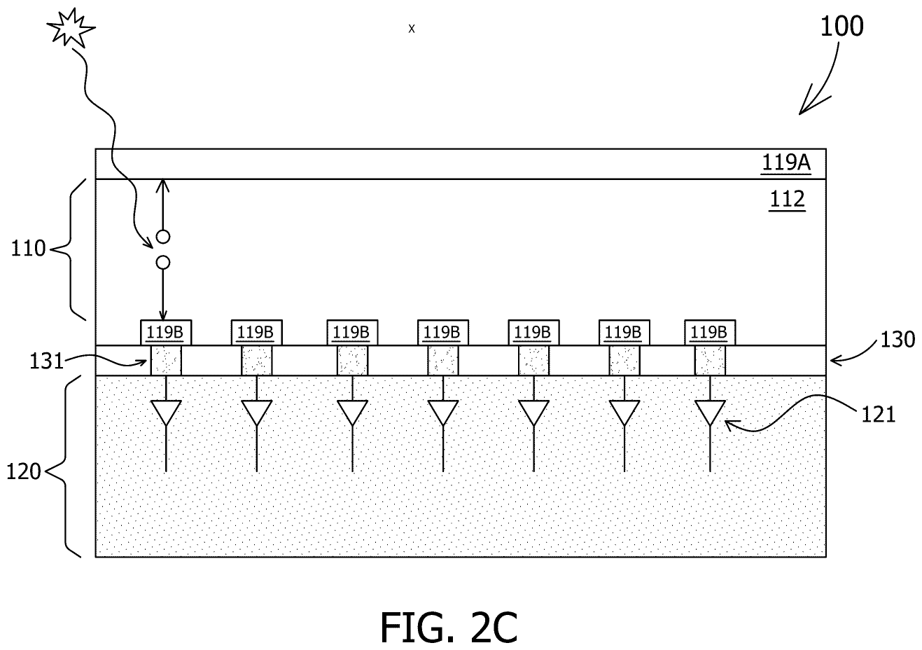

FIG. 2A schematically shows a simplified cross-sectional view of the radiation detector 100 of FIG. 1 along a line 2A-2A, according to an embodiment. More specifically, the radiation detector 100 may include a radiation absorption layer 110 and an electronics layer 120 (e.g., an ASIC) for processing or analyzing electrical signals which incident radiation generates in the radiation absorption layer 110. The radiation detector 100 may or may not include a scintillator (not shown). The radiation absorption layer 110 may include a semiconductor material such as, silicon, germanium, GaAs, CdTe, CdZnTe, or a combination thereof. The semiconductor material may have a high mass attenuation coefficient for the radiation of interest.

FIG. 2B schematically shows a detailed cross-sectional view of the radiation detector 100 of FIG. 1 along the line 2A-2A, as an example. More specifically, the radiation absorption layer 110 may include one or more diodes (e.g., p-i-n or p-n) formed by a first doped region 111, one or more discrete regions 114 of a second doped region 113. The second doped region 113 may be separated from the first doped region 111 by an optional intrinsic region 112. The discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example of FIG. 2B, each of the discrete regions 114 of the second doped region 113 forms a diode with the first doped region 111 and the optional intrinsic region 112. Namely, in the example in FIG. 2B, the radiation absorption layer 110 has a plurality of diodes (more specifically, 7 diodes corresponding to 7 pixels 150 of one row in the array of FIG. 1, of which only 2 pixels 150 are labeled in FIG. 2B for simplicity). The plurality of diodes have an electrode 119A as a shared (common) electrode. The first doped region 111 may also have discrete portions.

The electronics layer 120 may include an electronic system 121 suitable for processing or interpreting signals generated by the radiation incident on the radiation absorption layer 110. The electronic system 121 may include an analog circuitry such as a filter network, amplifiers, integrators, and comparators, or a digital circuitry such as a microprocessor, and memory. The electronic system 121 may include one or more ADCs. The electronic system 121 may include components shared by the pixels 150 or components dedicated to a single pixel 150. For example, the electronic system 121 may include an amplifier dedicated to each pixel 150 and a microprocessor shared among all the pixels 150. The electronic system 121 may be electrically connected to the pixels 150 by vias 131. Space among the vias may be filled with a filler material 130, which may increase the mechanical stability of the connection of the electronics layer 120 to the radiation absorption layer 110. Other bonding techniques are possible to connect the electronic system 121 to the pixels 150 without using the vias 131.

When radiation from the radiation source (not shown) hits the radiation absorption layer 110 including diodes, particles of the radiation may be absorbed and generate one or more charge carriers (e.g., electrons, holes) by a number of mechanisms. The charge carriers may drift to the electrodes of one of the diodes under an electric field. The field may be an external electric field. The electrical contact 119B may include discrete portions each of which is in electrical contact with the discrete regions 114. The term “electrical contact” may be used interchangeably with the word “electrode.” In an embodiment, the charge carriers may drift in directions such that the charge carriers generated by a single particle of the radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of the radiation incident around the footprint of one of these discrete regions 114 are not substantially shared with another of these discrete regions 114. A pixel 150 associated with a discrete region 114 may be an area around the discrete region 114 in which substantially all (more than 98%, more than 99.5%, more than 99.9%, or more than 99.99% of) charge carriers generated by a particle of the radiation incident therein flow to the discrete region 114. Namely, less than 2%, less than 1%, less than 0.1%, or less than 0.01% of these charge carriers flow beyond the pixel 150.

FIG. 2C schematically shows an alternative detailed cross-sectional view of the radiation detector 100 of FIG. 1 along the line 2A-2A, according to an embodiment. More specifically, the radiation absorption layer 110 may include a resistor of a semiconductor material such as, silicon, germanium, GaAs, CdTe, CdZnTe, or a combination thereof, but does not include a diode. The semiconductor material may have a high mass attenuation coefficient for the radiation of interest. In an embodiment, the electronics layer 120 of FIG. 2C is similar to the electronics layer 120 of FIG. 2B in terms of structure and function.

When the radiation hits the radiation absorption layer 110 including the resistor but not diodes, it may be absorbed and generate one or more charge carriers by a number of mechanisms. A particle of the radiation may generate 10 to 100,000 charge carriers. The charge carriers may drift to the electrical contacts 119A and 119B under an electric field. The electric field may be an external electric field. The electrical contact 119B includes discrete portions. In an embodiment, the charge carriers may drift in directions such that the charge carriers generated by a single particle of the radiation are not substantially shared by two different discrete portions of the electrical contact 119B (“not substantially shared” here means less than 2%, less than 0.5%, less than 0.1%, or less than 0.01% of these charge carriers flow to a different one of the discrete portions than the rest of the charge carriers). Charge carriers generated by a particle of the radiation incident around the footprint of one of these discrete portions of the electrical contact 119B are not substantially shared with another of these discrete portions of the electrical contact 119B. A pixel 150 associated with a discrete portion of the electrical contact 119B may be an area around the discrete portion in which substantially all (more than 98%, more than 99.5%, more than 99.9% or more than 99.99% of) charge carriers generated by a particle of the radiation incident therein flow to the discrete portion of the electrical contact 119B. Namely, less than 2%, less than 0.5%, less than 0.1%, or less than 0.01% of these charge carriers flow beyond the pixel associated with the one discrete portion of the electrical contact 119B.

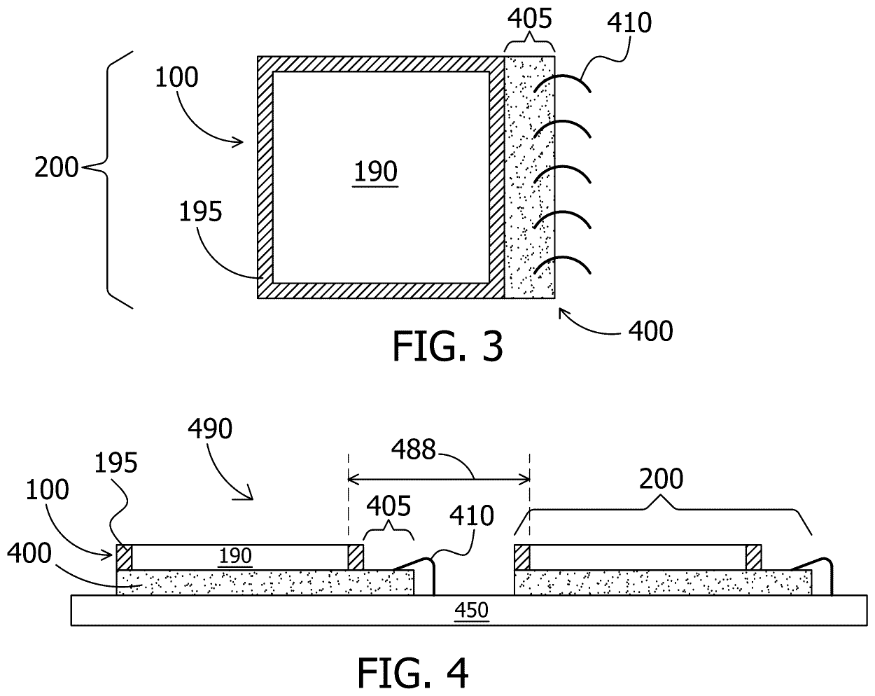

FIG. 3 schematically shows a top view of a package 200 including the radiation detector 100 and a printed circuit board (PCB) 400. The term “PCB” as used herein is not limited to a particular material. For example, a PCB may include a semiconductor. The radiation detector 100 may be mounted to the PCB 400. The wiring between the detector 100 and the PCB 400 is not shown for the sake of clarity. The PCB 400 may have one or more radiation detectors 100. The PCB 400 may have an area 405 not covered by the radiation detector 100 (e.g., for accommodating bonding wires 410). The radiation detector 100 may have an active area 190, which is where the pixels 150 (FIG. 1) are located. The radiation detector 100 may have a perimeter zone 195 near the edges of the radiation detector 100. The perimeter zone 195 has no pixels 150, and the radiation detector 100 does not detect particles of radiation incident on the perimeter zone 195.

FIG. 4 schematically shows a cross-sectional view of an image sensor 490, according to an embodiment. The image sensor 490 may include a plurality of the packages 200 of FIG. 3 mounted to a system PCB 450. FIG. 4 shows only 2 packages 200 as an example. The electrical connection between the PCBs 400 and the system PCB 450 may be made by bonding wires 410. In order to accommodate the bonding wires 410 on the PCB 400, the PCB 400 may have the area 405 not covered by the detector 100. In order to accommodate the bonding wires 410 on the system PCB 450, the packages 200 may have gaps in between. The gaps may be approximately 1 mm or more. Particles of radiation incident on the perimeter zones 195, on the area 405, or on the gaps cannot be detected by the packages 200 on the system PCB 450. A dead zone of a radiation detector (e.g., the radiation detector 100) is the area of the radiation-receiving surface of the radiation detector, on which incident particles of radiation cannot be detected by the radiation detector. A dead zone of a package (e.g., package 200) is the area of the radiation-receiving surface of the package, on which incident particles of radiation cannot be detected by the detector or detectors in the package. In this example shown in FIG. 3 and FIG. 4, the dead zone of the package 200 includes the perimeter zones 195 and the area 405. A dead zone (e.g., 488) of an image sensor (e.g., image sensor 490) with a group of packages (e.g., packages 200 mounted on the same PCB, packages 200 arranged in the same layer) includes the combination of the dead zones of the packages in the group and the gaps between the packages.

The image sensor 490 including the radiation detectors 100 may have the dead zone 488 incapable of detecting incident radiation. However, the image sensor 490 may capture partial images of all points of an object or scene (not shown), and then these captured partial images may be stitched to form a full image of the entire object or scene.

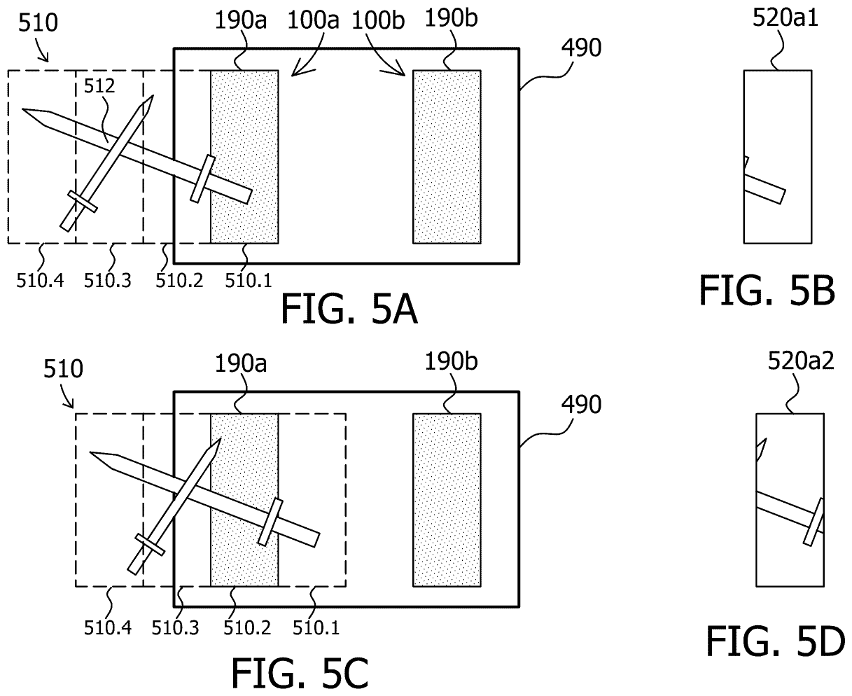

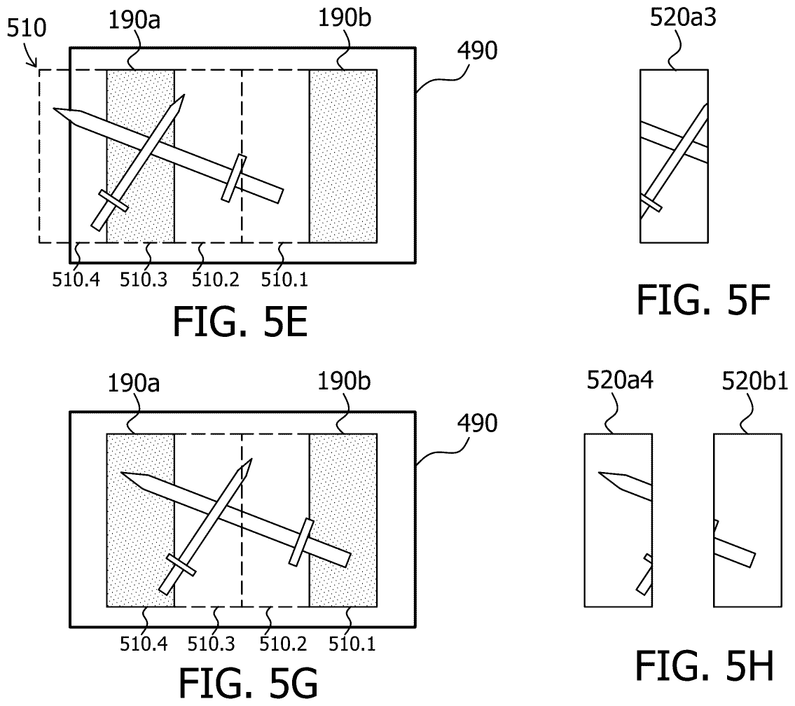

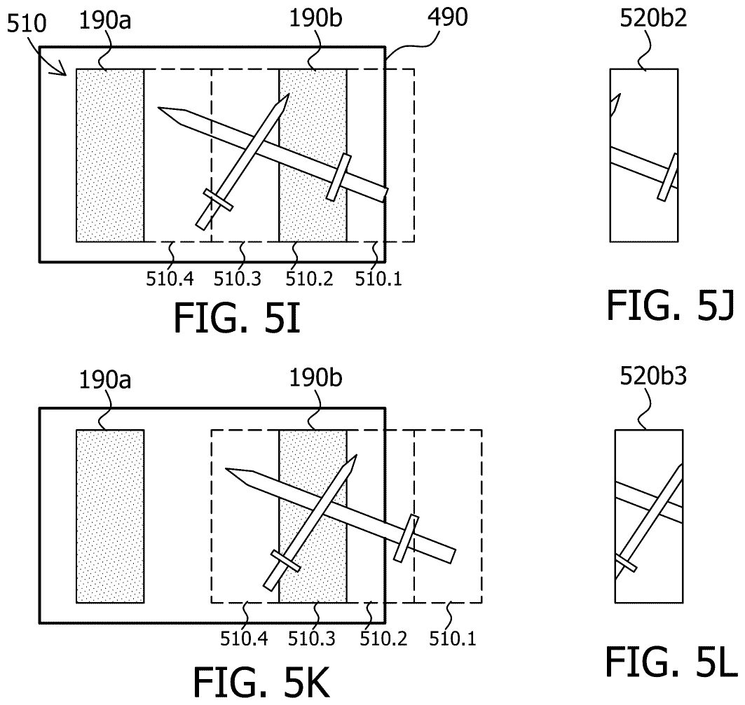

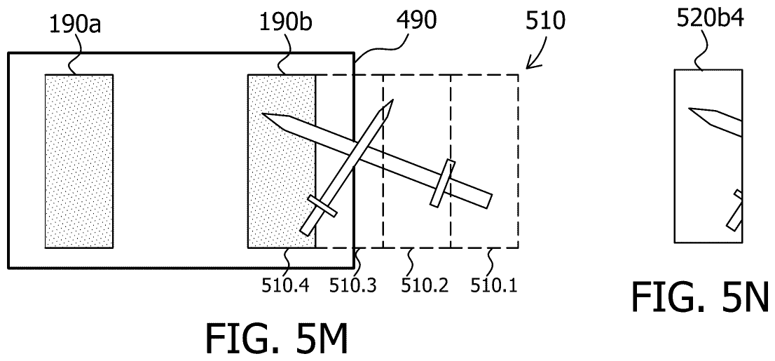

FIG. 5A-FIG. 5N schematically show an imaging session using the image sensor 490 of FIG. 4, according to an embodiment. With reference to FIG. 5A, in an embodiment, the image sensor 490 may be used to scan a scene 510. The image sensor 490 may include 2 radiation detectors 100a and 100b (similar to the radiation detector 100) which may include active areas 190a and 190b, respectively. For simplicity, only the active areas 190a and 190b of the image sensor 490 are shown whereas other parts of the image sensor 490 are omitted. In an embodiment, the radiation detectors 100a and 100b of the image sensor 490 may be identical.

For illustration, an object 512 (two swords) may be part of the scene 510. In an embodiment, the scene 510 may include 4 scene portions 510.1, 510.2, 510.3, and 510.4. In an embodiment, the scene 510 may be moved from left to right while the image sensor 490 remains stationary as the image sensor 490 scans the scene 510.

Specifically, in an embodiment, the scene 510 may start at a first imaging position (FIG. 5A) where the scene portion 510.1 is aligned with the active area 190a. In an embodiment, the active area 190a may capture a portion image 520a1 (FIG. 5B) of the scene portion 510.1 while the scene 510 remains stationary at the first imaging position.

Next, in an embodiment, the scene 510 may be moved further to the right to a second imaging position (FIG. 5C) where the scene portion 510.2 is aligned with the active area 190a. In an embodiment, the active area 190a may capture a portion image 520a2 (FIG. 5D) of the scene portion 510.2 while the scene 510 remains stationary at the second imaging position.

Next, in an embodiment, the scene 510 may be moved further to the right to a third imaging position (FIG. 5E) where the scene portion 510.3 is aligned with the active area 190a. In an embodiment, the active area 190a may capture a portion image 520a3 (FIG. 5F) of the scene portion 510.3 while the scene 510 remains stationary at the third imaging position.

Next, in an embodiment, the scene 510 may be moved further to the right to a fourth imaging position (FIG. 5G) where (A) the scene portion 510.4 is aligned with the active area 190a and (B) the scene portion 510.1 is aligned with the active area 190b. In an embodiment, the active area 190a and 190b may simultaneously capture portion images 520a4 and 520b1 (FIG. 5H) of the scene portions 510.4 and 510.1 respectively while the scene 510 remains stationary at the fourth imaging position.

Next, in an embodiment, the scene 510 may be moved further to the right to a fifth imaging position (FIG. 5I) where the scene portion 510.2 is aligned with the active area 190b. In an embodiment, the active area 190b may capture a portion image 520b2 (FIG. 5J) of the scene portion 510.2 while the scene 510 remains stationary at the fifth imaging position.

Next, in an embodiment, the scene 510 may be moved further to the right to a sixth imaging position (FIG. 5K) where the scene portion 510.3 is aligned with the active area 190b. In an embodiment, the active area 190b may capture a portion image 520b3 (FIG. 5L) of the scene portion 510.3 while the scene 510 remains stationary at the sixth imaging position.

Next, in an embodiment, the scene 510 may be moved further to the right to a seventh imaging position (FIG. 5M) where the scene portion 510.4 is aligned with the active area 190b. In an embodiment, the active area 190b may capture a portion image 520b4 (FIG. 5N) of the scene portion 510.4 while the scene 510 remains stationary at the seventh imaging position.

In summary of the imaging session described above, with reference to FIG. 5A-FIG. 5N, each of the active areas 190a and 190b scans through all the 4 scene portions 510.1, 510.2, 510.3, and 510.4. In other words, each of the scene portions 510.1, 510.2, 510.3, and 510.4 has images captured by both the active areas 190a and 190b. Specifically, the scene portion 510.1 has its images 520a1 and 520b1 captured by the active areas 190a and 190b respectively. The scene portion 510.2 has its images 520a2 and 520b2 captured by the active areas 190a and 190b respectively. The scene portion 510.3 has its images 520a3 and 520b3 captured by the active areas 190a and 190b respectively. The scene portion 510.4 has its images 520a4 and 520b4 captured by the active areas 190a and 190b respectively.

In an embodiment, with reference to FIG. 5A-FIG. 5N, for the scene portion 510.1, a first enhanced portion image (not shown) of the scene portion 510.1 may be generated from the portion images 520a1 and 520b1 of the scene portion 510.1. In an embodiment, the resolution of the first enhanced portion image may be higher than the resolutions of the portion images 520a1 and 520b1. For example, the resolution of the first enhanced portion image may be two times the resolutions of the portion images 520a1 and 520b1. Specifically, the portion images 520a1 and 520b1 each may have 28 picture elements (FIG. 1) whereas the first enhanced portion image may have 2×28=56 picture elements.

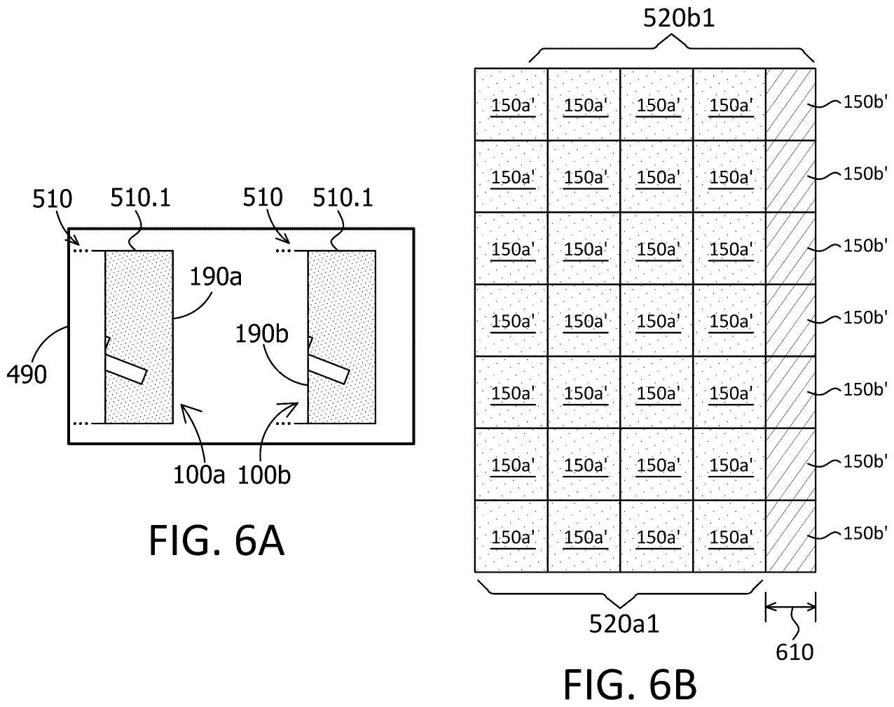

In an embodiment, the first enhanced portion image may be generated from the portion images 520a1 and 520b1 by applying one or more super resolution algorithms to the portion images 520a1 and 520b1. FIG. 6A and FIG. 6B show how one or more super resolution algorithms may be applied to the portion images 520a1 and 520b1 resulting in the first enhanced portion image, according to an embodiment.

Specifically, FIG. 6A shows the scene 510 at the first imaging position (left half of FIG. 6A, where the active area 190a captures the portion image 520a1 of the scene portion 510.1), and then later at the fourth imaging position (right half of FIG. 6A, where the active area 190b captures the portion image 520b1 of the scene portion 510.1). For simplicity, only the scene portion 510.1 of the scene 510 is shown (i.e., the other 3 scene portions 510.2, 510.3, and 510.4 of the scene 510 are not shown).

On one hand, in an embodiment, the positions and orientations of the radiation detectors 100a and 100b with respect to the image sensor 490 may be determined. From that, the displacement and relative orientation between the radiation detectors 100a and 100b with respect to the image sensor 490 may be determined. In an embodiment, these determinations may be performed by the manufacturer of the image sensor 490, and the resulting determination data may be stored in the image sensor 490 for later use in subsequent imaging sessions including the imaging session described above.

On the other hand, in an embodiment, during the imaging session described above, a step motor (not shown) may be used to move the scene 510 from the first imaging position through the second and third imaging positions to the fourth imaging position. In an embodiment, the step motor may include mechanism for measuring the distance of movement caused by the step motor. For example, electric pulses may be sent to the step motor so as to determine the displacement of the scene 510. As such, the displacement between the first imaging position and the fourth imaging position with respect to the image sensor 490 may be determined. Alternatively, instead of using a step motor with mechanism for measuring distance, optical diffraction may be used for determining the displacement between the first imaging position and the fourth imaging position with respect to the image sensor 490. In general, any method for determining the distance traveled by the scene 510 with respect to the image sensor 490 may be used for determining the displacement between the first imaging position and the fourth imaging position with respect to the image sensor 490.

As a simplified example, assume that the positions and orientations of the radiation detectors 100a and 100b with respect to the image sensor 490 are determined, and from that, (A) the displacement between the radiation detectors 100a and 100b is determined to be 12 sensing element widths (i.e., 12 times the width 102 of a sensing element 150 of FIG. 1) in the east direction, and (B) the relative orientation between the radiation detectors 100a and 100b is zero. In other words, the radiation detector 100a would need to translate in the east direction (no need to rotate) by a distance of 12 sensing element widths to reach and coincide with the radiation detector 100b.

Also in the simplified example, assume further that the displacement between the first imaging position and the fourth imaging position with respect to the image sensor 490 is determined to be 11.3 sensing element widths in the east direction. In other words, the scene 510 has moved in the east direction by a distance of 11.3 sensing element widths to reach the fourth imaging position.

As a result, in the simplified example, as shown in FIG. 6B, the 28 picture elements 150b′ of the portion image 520b1 are shifted to the right of the 28 picture elements 150a′ of the portion image 520a1 by an offset 610 of 0.7 (i.e., 12−11.3) sensing element width when the 2 portion images 520a1 and 520b1 are aligned such that the images of points of the scene portion 510.1 in the portion images 520a1 and 520b1 coincide. In FIG. 6B, for simplicity, the part of the portion image 520b1 that overlaps the portion image 520a1 is not shown.

In an embodiment, with the offset 610 determined (i.e., 0.7 sensing element width), one or more super resolution algorithms may be applied to the portion images 520a1 and 520b1 based on the determined offset 610, resulting in the first enhanced portion image of the scene portion 510.1.

Described above is the simplified example where the radiation detector 100a would need to translate in order to reach and coincide with the radiation detector 100b. In general, the radiation detector 100a might need to both translate and rotate in order to reach and coincide with the radiation detector 100b. This means that the orientations of the radiation detectors 100a and 100b with respect to the image sensor 490 are different, or in other words, the relative orientation between the radiation detectors 100a and 100b is different than zero.

In addition, in the general case, the displacement between the first imaging position and the fourth imaging position with respect to the image sensor 490 may be in a direction different than the east direction. However, in the general case, with sufficient information (i.e., (A) the positions and orientations of the radiation detectors 100a and 100b with respect to the image sensor 490 and (B) the displacement between the first and fourth imaging positions with respect to the image sensor 490), the portion images 520a1 and 520b1 may be aligned in a manner similar to the manner described above in the simplified example.

In summary, with the determination of the positions and orientations of the radiation detectors 100a and 100b with respect to the image sensor 490, and with the determination of the displacement between the first imaging position and the fourth imaging position with respect to the image sensor 490, the portion images 520a1 and 520b1 may be aligned, and the offset 610 between the picture elements 150a′ and 150b′ may be determined. As a result, one or more super resolution algorithms may be applied to the portion images 520a1 and 520b1 based on the determined offset 610 between the picture elements 150a′ and 150b′, resulting in the first enhanced portion image of the scene portion 510.1.

In an embodiment, a second enhanced portion image of the scene portion 510.2 may be generated from the portion images 520a2 and 520b2 in a similar manner; a third enhanced portion image of the scene portion 510.3 may be generated from the portion images 520a3 and 520b3 in a similar manner; and a fourth enhanced portion image of the scene portion 510.4 may be generated from the portion images 520a4 and 520b4 in a similar manner.

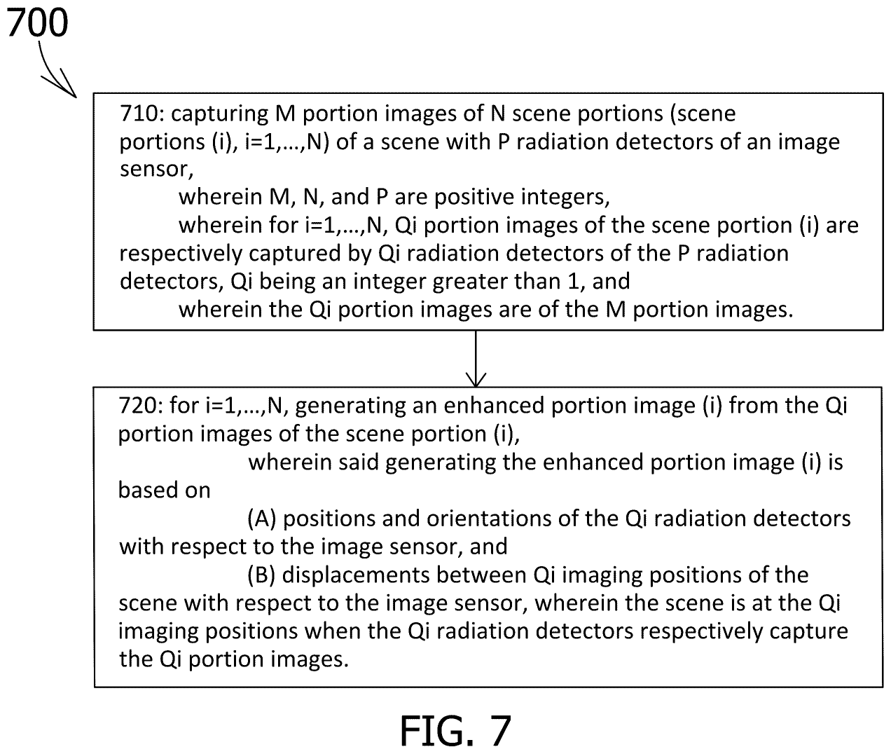

FIG. 7 is a flowchart 700 summarizing and generalizing the imaging session described above (FIG. 5A-FIG. 5N), according to an embodiment. Specifically, in step 710, M portion images (e.g., the M=8 portion images 520a1, 520a2, 520a3, 520a4, 520b1, 520b2, 520b3, and 520b4) of N scene portions (scene portions (i), i=1, . . . , N) (e.g., the N=4 scene portions 510.1, 510.2, 510.3, and 510.4) of a scene (e.g., the scene 510) are captured by P radiation detectors (e.g., the P=2 radiation detectors 100a and 100b) of an image sensor (e.g., the image sensor 490).

In addition, for i=1, . . . , N, Qi portion images (e.g., with i=1, the Q1=2 portion images 520a1 and 520b1) of the scene portion (i) (e.g., the scene portion 510.1) are respectively captured by Qi radiation detectors (e.g., the Q1=2 radiation detectors 100a and 100b) of the P radiation detectors. In addition, the Qi portion images (e.g., with i=1, the Q1=2 portion image 520a1 and 520b1) are of the M portion images (e.g., the M=8 portion images 520a1, 520a2, 520a3, 520a4, 520b1, 520b2, 520b3, and 520b4).

Next, in step 720, for i=1, . . . , N, an enhanced portion image (i) is generated (e.g., with i=1, the first enhanced portion image is generated) from the Qi portion images (e.g., from the Q1=2 portion images 520a1 and 520b1) of the scene portion (i) (e.g., the scene portion 510.1). In addition, the enhanced portion image (i) is generated based on (A) positions and orientations of the Qi radiation detectors (e.g., with i=1, the Q1=2 radiation detectors 100a and 100b) with respect to the image sensor, and (B) displacements between Qi imaging positions (e.g., the first and fourth imaging positions) of the scene (e.g., the scene 510) with respect to the image sensor, wherein the scene is at the Qi imaging positions when the Qi radiation detectors respectively capture the Qi portion images (e.g., the scene 510 is at the first and fourth imaging positions when the Q1=2 radiation detectors 100a and 100b respectively capture the Q1=2 portion images 520a1 and 520b1).

In an embodiment, with reference to the flowchart 700 of FIG. 7, at least 2 portion images of the M portion images are captured simultaneously by the image sensor. For example, with reference to FIG. 5G-FIG. 5H, the 2 portion images 520a4 and 520b1 are captured simultaneously by the 2 radiation detectors 100a and 100b, respectively.

In an embodiment, with reference to the flowchart 700 of FIG. 7, said capturing may include moving the scene on a straight line with respect to the image sensor throughout said capturing, wherein the scene does not reverse direction of movement throughout said capturing. For example, with reference to FIG. 5A-FIG. 5N, the scene 510 moves on a straight line in the east direction with respect to the image sensor 490 and does not move in the west direction at any time during the scanning of the scene 510.

In an embodiment, with reference to the flowchart 700 of FIG. 7, Qi may be equal to P for i=1, . . . , N. For example, in the imaging session described above, Q1=Q2=Q3=Q4=P=2. In other words, each of the 4 scene portions 510.1, 510.2, 510.3, and 510.4 is scanned by each of the P=2 radiation detectors 100a and 100b.

In an embodiment, the first, second, third, and fourth enhanced portion images may be stitched resulting a stitched image (now shown) of the scene 510 (FIG. 5A-FIG. 5M). In an embodiment, the stitching of the first, second, third, and fourth enhanced portion images may be based on the position and orientation of at least one of the radiation detectors 100a and 100b with respect to the image sensor 490. For example, the stitching of the first, second, third, and fourth enhanced portion images may based on the position and orientation of the radiation detector 100a.

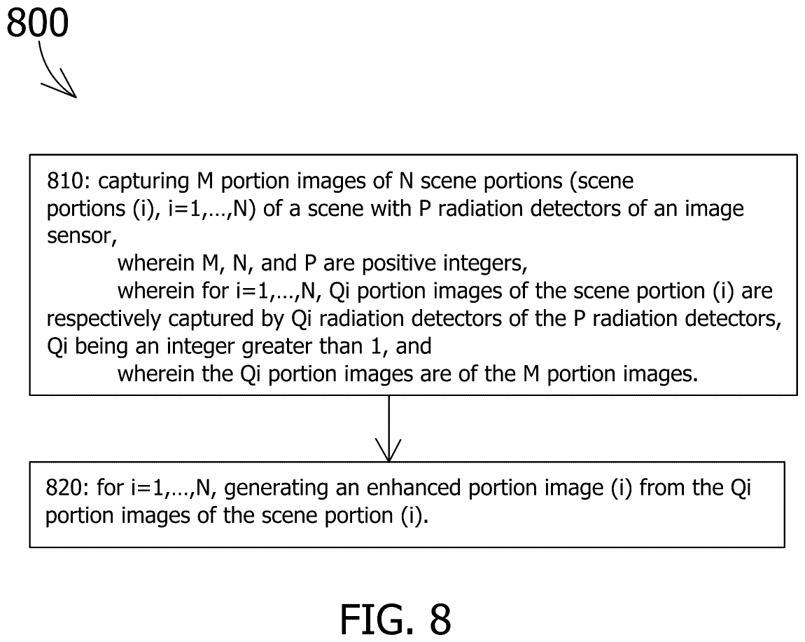

FIG. 8 is a flowchart 800 summarizing and generalizing the imaging session described above (FIG. 5A-FIG. 5N), according to an alternative embodiment. Specifically, in step 810, M portion images (e.g., the M=8 portion images 520a1, 520a2, 520a3, 520a4, 520b1, 520b2, 520b3, and 520b4) of N scene portions (scene portions (i), i=1, . . . , N) (e.g., the N=4 scene portions 510.1, 510.2, 510.3, and 510.4) of a scene (e.g., the scene 510) are captured with P radiation detectors (e.g., the P=2 radiation detectors 100a and 100b) of an image sensor (e.g., the image sensor 490).

In addition, for i=1, . . . , N, Qi portion images (e.g., with i=1, the Q1=2 portion images 520a1 and 520b1) of the scene portion (i) (e.g., the scene portion 510.1) are respectively captured by Qi radiation detectors (e.g., the Q1=2 radiation detectors 100a and 100b) of the P radiation detectors. In addition, the Qi portion images (e.g., with i=1, the Q1=2 portion image 520a1 and 520b1) are of the M portion images (e.g., the M=8 portion images 520a1, 520a2, 520a3, 520a4, 520b1, 520b2, 520b3, and 520b4).

Next, in step 820, for i=1, . . . , N, an enhanced portion image (i) is generated (e.g., with i=1, the first enhanced portion image is generated) from the Qi portion images (e.g., from the Q1=2 portion images 520a1 and 520b1) of the scene portion (i) (e.g., the scene portion 510.1).

In the embodiments described above, with reference to FIG. 5A-FIG. 5N, the image sensor 490 is kept stationary while the scene 510 (along with the object 512) is moved. Alternatively, the scene (along with the object 512) may be held stationary while the image sensor 490 (along with the radiation detectors 100a and 100b) may be moved as the image sensor 490 scans the scene 510.

In the embodiments described above, the image sensor 490 includes 2 radiation detectors 100a and 100b. In general, the image sensor 490 may have any number of the radiation detectors 100. In addition, each of the 4 scene portions 510.1, 510.2, 510.3, and 510.4 does not necessarily have its images captured by all the radiation detectors of the image sensor 490. Moreover, each of the 4 scene portions 510.1, 510.2, 510.3, and 510.4 does not necessarily have its images captured by the same radiation detectors.

For example, assume the image sensor 490 includes radiation detectors 100a, 100b, and a third radiation detector (not shown, but similar to the radiation detector 100). Then, in an embodiment, the scene portion 510.1 may have its 2 images captured respectively by the radiation detectors 100a and 100b; the scene portion 510.2 may have its 2 images captured respectively by the radiation detector 100a and the third radiation detector; the scene portion 510.3 may have its 2 images captured respectively by the radiation detector 100b and the third radiation detector; and the scene portion 510.4 may have its 3 images captured respectively by all the radiation detectors (100a, 100b, and the third radiation detector).

In the embodiments described above, the positions and orientations of the radiation detectors 100a and 100b with respect to the image sensor 490 are used to help align the portion images 520a1 and 520b1 (FIG. 7, step 720, part (A)). Alternatively, the displacement and relative orientation between the radiation detectors 100a and 100b with respect to the image sensor 490 may be used in place of the positions and orientations of the radiation detectors 100a and 100b to help align the portion images 520a1 and 520b1. Specifically, as shown in the simplified example described above, the displacement of 12 sensing element widths in the east direction between the radiation detectors 100a and 100b with respect to the image sensor 490 and the relative orientation of zero between the radiation detectors 100a and 100b are used to help determine the offset 610 (i.e., to help align the portion images 520a1 and 520b1).

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method, comprising:
capturing M portion images of N scene portions (scene portions (i), i=1, . . . , N) of a scene with P radiation detectors of an image sensor,
wherein M, N, and P are positive integers, and
wherein for i=1, . . . , N, Qi portion images of the scene portion (i) are respectively captured by Qi radiation detectors of the P radiation detectors, Qi being an integer greater than 1, and
wherein the Qi portion images are of the M portion images; and
for i=1, . . . , N, generating an enhanced portion image (i) from the Qi portion images of the scene portion (i),
wherein said generating the enhanced portion image (i) is based on
(A) positions and orientations of the Qi radiation detectors with respect to the image sensor, and
(B) displacements between Qi imaging positions of the scene with respect to the image sensor, wherein the scene is at the Qi imaging positions when the Qi radiation detectors respectively capture the Qi portion images.

2. The method of claim 1, wherein at least 2 portion images of the M portion images are captured simultaneously by the image sensor.

3. The method of claim 2, wherein said at least 2 portion images are captured by at least 2 radiation detectors of the P radiation detectors.

4. The method of claim 1, wherein for i=1, . . . , N, Qi>2.

5. The method of claim 1, wherein N>1.

6. The method of claim 1, wherein Qi=P for i=1, . . . , N.

7. The method of claim 1, wherein said generating the enhanced portion image (i) comprises applying one or more super resolution algorithms to the Qi portion images.

8. The method of claim 7, wherein said applying the one or more super resolution algorithms to the Qi portion images comprises aligning the Qi portion images.

9. The method of claim 1, further comprising stitching the enhanced portion images (i), i=1, . . . , N resulting in a stitched image of the scene.

10. The method of claim 9, wherein said stitching is based on a position and an orientation of at least one of the P radiation detectors with respect to the image sensor.

11. The method of claim 1, further comprising determining said displacements between the Qi imaging positions with a step motor which comprises mechanism for measuring a distance of movement caused by the step motor.

12. The method of claim 1, further comprising determining said displacements between the Qi imaging positions with optical diffraction.

13. The method of claim 1,
wherein said capturing comprises moving the scene on a straight line with respect to the image sensor throughout said capturing.

14. The method of claim 13, wherein the scene does not reverse direction of movement throughout said capturing.

15. The method of claim 1, wherein N>1, wherein j and k belong to 1, . . . , N, wherein j≠k, and wherein the Qj radiation detectors are different than the Qk radiation detectors.

16. The method of claim 1, wherein N>1, wherein j and k belong to 1, . . . , N, wherein j≠k, and wherein Qj≠Qk.

17. A method, comprising:
capturing M portion images of N scene portions (scene portions (i), i=1, . . . , N) of a scene with P radiation detectors of an image sensor,
wherein M, N, and P are positive integers, and
wherein for i=1, . . . , N, Qi portion images of the scene portion (i) are respectively captured by Qi radiation detectors of the P radiation detectors, Qi being an integer greater than 1, and
wherein the Qi portion images are of the M portion images; and
for i=1, . . . , N, generating an enhanced portion image (i) from the Qi portion images of the scene portion (i).

18. The method of claim 17, wherein said generating the enhanced portion image (i) is based on
(A) displacements and relative orientations between the Qi radiation detectors with respect to the image sensor, and
(B) displacements between Qi imaging positions of the scene with respect to the image sensor, wherein the scene is at the Qi imaging positions when the Qi radiation detectors respectively capture the Qi portion images.

19. The method of claim 17, wherein at least 2 portion images of the M portion images are captured simultaneously by the image sensor.

20. The method of claim 19, wherein said at least 2 portion images are captured by at least 2 radiation detectors of the P radiation detectors.
